(19) United States
(12) Patent Application Publication
(10) Pub. No.: US 2018/0338918 A1
(43) Pub. Date: Nov. 29, 2018

(54) TEMSIROLIMUS LIPOSOME AND PREPARATION METHOD THEREOF

(71) Applicant: JIANGSU TASLY DIYI PHARMACEUTICAL CO., LTD., Huai'an (CN)

(72) Inventors: Guocheng WANG, Tianjin (CN); Jianming CHEN, Shanghai (CN); Qinqin ZHOU, Shanghai (CN); Bao'an GAO, Shanghai (CN)

(21) Appl. No.: 15/985,479

(22) Filed: May 21, 2018

(30) Foreign Application Priority Data
May 24, 2017 (CN) 201710375571.7

(51) Int. Cl. A61K 9/127; A61K 31/436; A61K 47/26

(52) U.S. Cl. CPC A61K 9/1271; A61K 47/26; A61K 31/436; A61K 9/1277

(57) ABSTRACT

A temsirolimus liposome and preparation method thereof is provided. The formulation of the present invention contains temsirolimus, a phospholipid, a PEGylated phospholipid, and can further contain cholesterol, a stabilizer and a lyoprotectant. In view of the unique physicochemical properties of temsirolimus, the present invention performed a matching study on the formulation composition and preparation process, and developed a temsirolimus liposome, preferably a lyophilized powder injection, which is safe, stable in quality, simple in preparation process, and can be industrially produced. The preparation overcomes the disadvantages of poor safety, stability and the like in the existing preparations, establishing a solid foundation for the further study and application of temsirolimus in the anti-tumor field.

### FIELD OF THE INVENTION

The present invention relates to the field of medicinal technology, and in particular to a temsirolimus liposome and preparation method thereof.

### BACKGROUND OF THE INVENTION

Temsirolimus is the 42-bis(hydroxymethyl) propionate of sirolimus, and the structural formula thereof is shown below. Temsirolimus is almost insoluble in water, and as a non-electrolyte, the solubility thereof cannot be increased via pH adjustment, salification and other methods. Although the solubility thereof in some pharmaceutically acceptable organic solvents (such as ethanol, propanediol and polyethylene glycol, etc.) is better, almost all of them brought about the chemical stability problems such as readily oxidative degradation and cleavage of lactone ring; on the other hand, when temsirolimus is dissolved in the above organic solvents, it cannot be directly diluted with a 0.9% sodium chloride solution or 5% glucose solution and other aqueous solutions during clinical use, or else it will precipitate out and cannot be used clinically. Due to the above reasons, there are great difficulties in formulating temsirolimus as drugs.

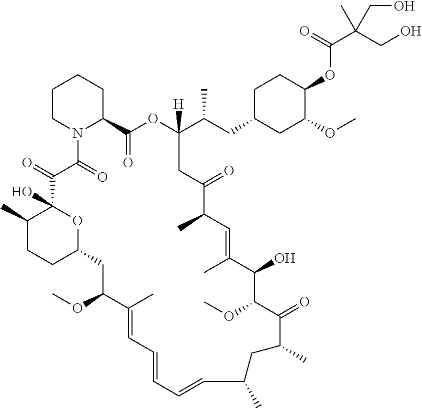 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

Studies have shown that temsirolimus exhibits a significant inhibitory effect, rather than cytotoxicity, on tumor growth in both of in vivo and in vitro models, and can delay tumor progression or recurrence. Its action mechanism, which is similar to that of sirolimus, involves binding to the cytoplasmic protein FKBP and forming a complex, thereby inhibiting the enzyme mTOR (the mammalian target of rapamycin). Inhibition of mTOR kinase activity results in the suppression of several signaling pathways, including cytokine-stimulated cell proliferation, mRNA translation for various key proteins (which regulating the G1 phase of the cell cycle) and IL-2 induced transcription, thereby inhibiting the process of the cell cycle from G1 to S phase. Such an action mechanism of arresting the process from G1 to S phase exerted by temsirolimus is a new mechanism for anti-cancer drugs.

Due to its advantages in tumor inhibition, temsirolimus is the first anti-tumor mTOR inhibitor approved by the U.S. FDA, which is an orphan drug for the treatment of advanced renal cell carcinoma. Wyeth LLC. solved the solubility problem of temsirolimus by employing a large amount of organic vehicles, and simultaneously solved the precipitation problem upon directly diluting drugs with aqueous solutions by adopting a specific diluent containing organic solvents and/or Tween 80, thereby developing an two-bottle preparation of temsirolimus, including a temsirolimus concentrate and diluent (TORISEL®). It was approved by the U.S. FDA on May, 2007 for use in advanced renal cancer, and approved by the European Union for use in the first-line treatment of advanced renal carcinoma at least having three or more prognostic risk factors. In this preparation, the compositions of the temsirolimus concentrate are temsirolimus, absolute ethanol, dl-α-tocopherol, propanediol and anhydrous citric acid; the compositions of the diluent are Tween 80 polyethylene glycol 400 and absolute ethanol. However, this preparation also has many disadvantages: \textcircled{1} the preparation contains a large amount of organic vehicles, which have strong irritability when being used for intravenous administration; \textcircled{2} the preparation contains Tween 80, which may cause an allergic reaction, and the package insert clearly indicates that antihistamine agents must be given prior to administration; \textcircled{3} since Tween 80 is contained therein, which can dissolve the plasticizer (Bis(2-ethylhexyl) phthalate) contained in infusion bags and tubes, the compatibility of the preparation and the infusion sets is poor; \textcircled{4} the preparation is a two-bottle preparation, which must be firstly diluted with the special diluent and then with physiological saline, otherwise precipitates is produced. This two-step dilution is operated complicatedly and also increases the risk of causing secondary pollution.

In view of said many deficiencies of the marketed temsirolimus preparation, there is an urgent need to develop a new, safe and stable temsirolimus preparation. Related reports on new temsirolimus preparations are currently limited. Patent application CN200480021450.3 discloses a lyophilized CCI-779 (temsirolimus) formulation. The main difference between this preparation and the marketed TORISEL® was that the temsirolimus solution was lyophilized to increase the stability thereof. However, when administered parenterally, it still needs to be firstly dissolved with a specific diluent containing organic solvents and/or Tween 80, and then diluted with physiological saline and other aqueous solutions for use. Patent application CN201210460639.9 discloses a lyophilized preparation of temsirolimus, which can be directly formulated for use with water for injection or physiological saline by adding a cosolvent polyethylene glycol stearate 15, solving the problems of complicated operation, easily causing secondary pollution and the like in TORISEL® and the preparation disclosed in patent application CN200480021450.3, which need to be bottles in two bottles, and must be firstly diluted with a special diluent and then with physiological saline. However, this preparation applied a large amount of surfactant polyethylene glycol stearate 15 to facilitate dissolution. The literature (Jia Zhang, Yikui Li, Lianda Li, et al., Acute toxicity of Tween-80, Poloxamer 188 and other two solubilizers in mice, Chinese Journal of New Drugs, 2008, 17(17): 1491-1493) reports that the acute toxicity of this surfactant injected via tail vein of mice was great, even greater than that of Tween 80, showing that the safety issue of this surfactant was still debatable. The literature (Haixia Ma, Xiang Zou, Optimization on preparation condition of temsirolimus liposome by response surface methodology, National Academic Conference on Antibiotics, 2013) prepared a temsirolimus liposome, but its encapsulation efficiency was only about 83%. The literature (Ran Mo, Qiong Sun, Nan Li, et al., Intracellular delivery and antitumor effects of pH-sensitive liposomes based on zwitterionic oligopeptide lipids, Biomaterials, 2013, 34(11): 2773-2786.) prepares a temsirolimus pH-sensitive liposome by adding a zwitterionic oligopeptide HHG2C₁₈ into a liposomal membrane material. Firstly, the study results of this literature show that when HHG2C₁₈ or PEGHG2C₁₈ were added to the liposomal membrane material, the prepared bare liposome and drug-loaded liposome both were significantly increased in particle sizes. Especially after the addition of PEGHG2C₁₈, the loading capacity on the drug temsirolimus was also significantly decreased. It can be seen that this component has a certain influence on the druggability of liposome, and may affect the stability of liposome and the encapsulation of drugs. Moreover, according to the structure of this component, the hydrophobic portion thereof was divided into two stearyl alkane chains, and its affinity for phospholipids was obviously inferior to PEGylated phospholipids (i.e., PEG-modified phospholipids). If used as liposomal membrane components, it was likely to be detrimental to the formation of liposomes and/or the encapsulation of drugs. Secondly, the literature (Chao Fang, Bin Shi, Ming Hong, et al., Influence of particle size and MePEG molecular weight on in vitro macrophage uptake and in vivo long circulating of stealth nanoparticles in rats, Acta Pharmaceutica Sinica, 2006, 41(4):305-312.) compares the influences of 80 nm, 170 nm, and 240 nm particle sizes on in vitro macrophage uptake and in vivo long circulating in rats, respectively. The results show that the uptake by macrophages decreased and the plasma half-life in rats prolonged with the decrease of particle size. It can be seen that when PEGHG2C₁₈ was added into the liposomal membrane material, the particle size of the liposome increased from 100 nm of the original common liposome to 150 nm, which may has an adverse effect on macrophage uptake, in vivo circulation time and the like.

Therefore, in view of the existing deficiencies in the current temsirolimus preparations, we screened some common safer dosage forms for temsirolimus, such as sulfobutyl ether-β-cyclodextrin inclusion complex, liposome, and fat emulsion, expecting to find a dosage form matched with temsirolimus (see Example 1). The results show that the druggabilities of sulfobutyl ether-β-cyclodextrin and fat emulsion for temsirolimus were extremely low, mainly reflected in that: (1) up to 50% of sulfobutyl ether-β-cyclodextrin still failed to achieve the inclusion of 1 mg/ml temsirolimus; (2) the solubility of temsirolimus in common oils for injection (medium chain oil, soybean oil) was too low to meet the requirement of preparation. For the liposome prepared with conventional formulation process, the quality thereof was poor, which was similar to the temsirolimus liposome mentioned in the above literatures. However, compared to the two dosage forms of sulfobutyl ether-β-cyclodextrin and fat emulsion, the druggability of liposome was better. After a large number of experiments, we found that the addition of quantum satis PEGylated phospholipids into the membrane material of common liposome can significantly improve the quality of temsirolimus liposome. By comparing the encapsulation efficiency and stability of temsirolimus liposome with and without PEGylated phospholipids, we found that the addition of PEGylated phospholipids can significantly improve the encapsulation efficiency (>90%) and stability (see Example 2), which may be related to the unique physicochemical properties of temsirolimus.

In view of the above problems, in combination with the physicochemical properties of temsirolimus, the present invention successfully developed a temsirolimus liposome, which is safe, high encapsulation efficiency and stable in quality, through a large number of experimental studies, solved the existing deficiencies in the available temsirolimus preparations, and provided a new preparation for clinical study and application.

### SUMMARY OF THE INVENTION

The object of the present invention is to provide a temsirolimus liposome which is safe, high encapsulation efficiencies and stable in quality. Another object of the present invention is to provide a preparation method of temsirolimus liposome.

In view of the existing deficiencies in the available temsirolimus preparations, we aimed to develop a preparation that is free of Tween 80, single-packaged, stable and safe. In combination with the physicochemical properties of temsirolimus, the cyclodextrin inclusion complex, common nano-encapsulation preparation (e.g., liposome, fat emulsion) and other dosage forms were investigated (See Example 1). Surprisingly, only liposome has a certain practicability, while other dosage forms failed to show any signs of being implementable. For example, the solubility of temsirolimus in common oils for injection (medium chain oil, soybean oil) was very low, and temsirolimus was still in a state of severe turbidity at 10 mg/g; when sulfobutyl ether-β-cyclodextrin was used for inclusion, even if the application amount thereof reached 50%, the inclusion on 1 mg/ml of temsirolimus cannot be achieved. When conventional formulation compositions and dosages of liposome were utilized, although liposome can be barely obtained for temsirolimus, the quality was far from good, and a series of problems such as low encapsulation efficiency, drug leakage, large insoluble microparticles, poor stability and the like existed. During the experiment, it was unexpectedly discovered that if PEGylated phospholipids were added into the formulation and used together with lecithin as encapsulating materials, quite well effects were achieved and the above problems were solved perfectly. This discovery may be related with the physicochemical properties of temsirolimus, and liposome in compliance with requirements can be successfully prepared only if it is consistent with the inherent characteristics of temsirolimus. Therefore, the temsirolimus liposome studied in the present invention contains PEGylated phospholipids in formulation, which is one of the core technical features of the present invention.

In one aspect of the present invention, a temsirolimus liposome is provided, which further contains PEGylated phospholipids in addition to temsirolimus and phospholipids, to increase the encapsulation efficiency and stability of the liposome.

Therefore, the present invention provides a temsirolimus liposome composition, formulated by the following components in weight ratios:

| | | |
|---|---|---|
| Temsirolimus | 0.5-10 | parts |
| Phospholipid | 10-100 | parts |
| PEGylated phospholipid | 0.2-10 | parts |
| Cholesterol | 0-10 | parts |
| Stabilizer | 0-10 | parts |
| Lyoprotectant | 50-400 | parts |

| | |
|---|---|
| Vehicle | q.s. |

Preferably, the liposome composition of the present invention is formulated by the following components in weight ratios:

| | | |
|---|---|---|
| Temsirolimus | 0.5-7 | parts |
| Phospholipid | 15-80 | parts |
| PEGylated phospholipid | 0.5-8 | parts |
| Cholesterol | 0-5 | parts |
| Stabilizer | 0.05-8 | parts |
| Lyoprotectant | 100-400 | parts |

| | |
|---|---|
| Vehicle | q.s. |

More preferably, the liposome composition of the present invention is formulated by the following components in weight ratios:

| | | |
|---|---|---|
| Temsirolimus | 1-5 | parts |
| Phospholipid | 15-60 | parts |
| PEGylated phospholipid | 1-5 | parts |
| Cholesterol | 0-4 | parts |
| Stabilizer | 0.1-5 | parts |
| Lyoprotectant | 100-350 | parts |

| | |
|---|---|
| Vehicle | q.s. |

Wherein, the vehicle includes the organic vehicles or water used during the preparation of the liposome composition, which are used during the preparation, but dried and removed in the final product. The “q.s. (quantum satis)” means different amounts used according to different components, with the essential requirements of being able to dissolve relevant components. The amounts used belong to conventional techniques in the art and are not essential technical features of the present invention.

Wherein, the phospholipid is selected from one or two or more of high-purity egg yolk lecithin, high-purity soybean lecithin, hydrogenated soybean lecithin, hydrogenated egg yolk lecithin, dipalmitoyl phosphatidylcholine (DPPC), distearoyl phosphatidylcholine (DSPC), dierucoyl phosphatidylcholine (DEPC), dioleoyl phosphatidylcholine (DOPC), dimyristoyl phosphatidylcholine (DMPC), 1-palmitoyl-2-oleoyl-sn-glycero-3-phosphocholine (POPC), phosphatidylcholine, egg yolk lecithin, soybean lecithin, phosphatidylserine, phosphatidyl ethanolamine and sphingomyelin, preferably high-purity egg yolk lecithin.

Wherein, the PEGylated phospholipid is selected from 1,2-distearoyl-sn-glycero-3-phosphoethanolamine-N-[methoxy(polyethylene glycol)] (DSPE-PEG) and/or dipalmitoyl phosphoethanolamine-PEG (DPPE-PEG), preferably 1,2-distearoyl-sn-glycero-3-phosphoethanolamine-N-[methoxy(polyethylene glycol)] (DSPE-PEG); wherein the molecular weight of polyethylene glycol is selected from 1000-8000, preferably 2000-5000. Wherein, the stabilizer comprises a chelating agent and/or an antioxidant selected from one or two or more of ethylenediamine tetraacetic acid and salts thereof, α-tocopherol, α-tocopheryl succinate, α-tocopheryl acetate, citric acid, glycine, glutamic acid, succinic acid, adipic acid, malic acid, maleic acid, ascorbic acid, sodium sulfite, sodium bisulfite, sodium metabisulfite, glutathione, cysteine, thioglycerol, tert-butyl hydroxyanisole, di-tert-butyl p-cresol and propyl gallate, preferably α-tocopherol, ethylenediamine tetraacetic acid and salts thereof.

Wherein, the lyoprotectant is selected from one or two or more of maltose, trehalose, sucrose, mannitol, lactose, glucose, sorbitol, xylitol, erythritol, threonine, preferably sucrose, maltose and trehalose.

In the liposome composition according to the present invention, 0-40 parts of propanediol, a cosolvent, may further be added as required.

The present invention further provides a preparation method of the liposome composition according to the present invention, and the method can be the following method:

taking temsirolimus, a phospholipid, a PEGylated phospholipid, cholesterol and a stabilizer, adding quantum satis organic vehicle thereto, and dissolving them by heating at 25-75° C. to give an organic phase; weighing quantum satis water for injection, and heating it to 25-75° C., so as to give an aqueous phase; pouring the organic phase into the aqueous phase with stirring, and mixing uniformly to give a crude liposome; emulsifying the crude liposome, which can be homogenized and emulsified by placing it in a high-pressure homogenizer, or extruded successively through extrusion membranes with different pore sizes by placing it in an extruder, or extruded after high-pressure homogenization, so as to give a liposome solution; weighing a formula amount of a lyoprotectant, placing it in the liposome solution, dissolving it with stirring, and setting the volume thereof to full volume with water for injection; adjusting pH value with a pH regulator; filtering and sterilizing through a 0.22 μm filter membrane, sub-packaging, lyophilizing and sealing, and thus obtaining temsirolimus liposome lyophilized powder injection, wherein the organic vehicle is selected from one or two or more of anhydrous ethanol, propanediol and tert-butanol.

The method can also be the following method:

taking temsirolimus, a phospholipid, a PEGylated phospholipid, cholesterol and a stabilizer, adding quantum satis organic vehicle thereto, and dissolving them by heating at 25-75° C. to give an organic phase; subjecting the organic phase to a rotary evaporation to remove the organic vehicle at 25-75° C., or placing the organic phase in a sample plate and lyophilizing it to give a lipid phase; weighing quantum satis water for injection and heating it to 25-75° C., giving an aqueous phase; adding the aqueous phase to the lipid phase, and stirring them to give a crude liposome; emulsifying the crude liposome, which can be homogenized and emulsified by placing it in a high-pressure homogenizer, or extruded successively through extrusion membranes with different pore sizes by placing it in an extruder, or extruded after high-pressure homogenization, so as to give a liposome solution; weighing a formula amount of a lyoprotectant, placing it in the liposome solution, dissolving it with stirring and setting the volume thereof to full volume with water for injection; adjusting pH value with a pH regulator; filtering and sterilizing through a 0.22 μm filter membrane, sub-packaging, lyophilizing and sealing, and thus obtaining temsirolimus liposome lyophilized powder injection, wherein the organic vehicle is selected from one or two or more of ethanol, dichloromethane, chloroform, methanol, acetonitrile, tert-butanol, ethylpropanediol and methanol. The stabilizer can also be dissolved in the aqueous phase depending on the solubility property thereof.

The lyoprotectant can also be added to the aqueous phase.

The crude liposome is emulsified, and the emulsification method is preferably an extrusion emulsification method. The pore size of the extrusion membrane is selected from 2.0 μm, 1.0 μm, 0.8 μm, 0.6 μm, 0.4 μm, 0.2 μm, 0.1 μm and 0.05 μm. One or two or more pore sizes are selected for successive extrusion from large pore sizes to small pore sizes.

The pH regulator described in the above method is selected from organic or inorganic acids or bases selected from one or two or more of citric acid, acetic acid, propionic acid, hydrochloric acid, sodium hydroxide, phosphoric acid, triethylamine, dipotassium hydrogen phosphate, potassium dihydrogen phosphate, disodium hydrogen phosphate, sodium dihydrogen phosphate, sodium citrate and sodium acetate.

The pH regulator adjusts the pH value, and adjusting the pH value to 3-8; preferably to 4-7; more preferably to 4-6.

The temsirolimus liposome of the present invention has a particle size of 70-140 nm.

The advantages of the present invention lie in:

(1) The temsirolimus liposome of the present invention is free of Tween 80, polyoxyethylenated castor oil and other solubilizers, and avoids the allergic reaction caused by Tween 80 and other solubilizers in the marketed preparation TORISEL®.
(2) The temsirolimus liposome of the present invention belongs to a nano-encapsulation preparation with a particle size of about 100 nm, has an EPR effect, and has a certain targeting effect on tumor sites.
(3) Through optimization of the formulation process, the temsirolimus liposome of the present invention has the characteristics of high encapsulation efficiency, good stability and the like, and further increases the chemical stability of temsirolimus by preparing it into the lyophilized preparation.
(4) The temsirolimus liposome of the present invention has a simple preparation process, can realize industrial production, and is convenient for clinical use without the need of diluting it with different diluents for several times.

### DETAILED EMBODIMENTS OF THE INVENTION

Hereinafter, the present invention will be further illustrated in combination with specific Examples. It should be understood that the following Examples are used for illustrating the present invention only, instead of limiting the scope of the present invention.

### Example 1: Investigation of Different Dosage Forms on the Druggability of Temsirolimus

The druggabilities of sulfobutyl ether-β-cyclodextrin inclusion complex, liposome, and fat emulsion were investigated. The drug loading was fixed at 1 mg/ml for parallel comparisons. The main study protocol and results were summarized as follows:

### 1. Sulfobutyl Ether-β-Cyclodextrin Inclusion Complex

### 1.1 Formulation Process 1

### 1.1.1 Formulation

| | | Sulfobutyl | Water for |
|---|---|---|---|
| | Temsirolimus | ether-β-cyclodextrin | injection |
| Formulation 1 | 100 mg | 10 g | to 100 mL |
| Formulation 2 | 100 mg | 30 g | to 100 mL |
| Formulation 3 | 100 mg | 50 g | to 100 mL |

### 1.1.2 Preparation Method

Formula amount of sulfobutyl ether-β-cyclodextrin was weighed, and dissolved by adding quantum satis water for injection, and setting the volume thereof to 100 mL; formula amount of temsirolimus was weighed, added into the aqueous solution of sulfobutyl ether-β-cyclodextrin, and stirred them at room temperature for 2 h.

### 1.1.3 Results Evaluation

After being stirred for 2 h, each of the above formulations exhibited a state of severe turbidity.

### 1.2 Formulation Process 2

### 1.2.1 Formulation

| | | Anhydrous | | | Sulfobutyl | Water for |
|---|---|---|---|---|---|---|
| | Temsirolimus | ethanol | Propanediol | PEG4000 | ether-β-cyclodextrin | injection |
| Formulation 1 | 100 mg | 4 g | / | / | 50 g | to 100 mL |
| Formulation 2 | 100 mg | / | 4 g | / | 50 g | to 100 mL |
| Formulation 3 | 100 mg | / | / | 4 g | 50 g | to 100 mL |

### 1.2.2 Preparation Method

Formula amount of sulfobutyl ether-β-cyclodextrin was weighed, dissolved by adding quantum satis water for injection to give an aqueous phase; formula amounts of temsirolimus and organic vehicle (anhydrous ethanol, or propanediol, or PEG4000) were weighed, dissolved by water-bath ultrasound to give an organic phase; the organic phase was poured into the aqueous phase with stirring, and mixed uniformly, and setting the volume thereof to full volume with water for injection, and thus the formulations were obtained.

### 1.2.3 Results Evaluation

With the addition of the drug solution into the aqueous solution of sulfobutyl ether-β-cyclodextrin, the solution of each formulation gradually becomes turbid, wherein the degree of turbidity, when using anhydrous ethanol as vehicle, was greater than that of propanediol and PEG400.

In summary, sulfobutyl ether-β-cyclodextrin can hardly achieve the inclusion of temsirolimus.

### 2. Liposome

### 2.1 Formulation

| | Formu- | | Formu- | | Formu- | | Formu- | |
|---|---|---|---|---|---|---|---|---|
| | lation 1 | | lation 2 | | lation 3 | | lation 4 | |
| Temsirolimus | 100 | mg | 100 | mg | 100 | mg | 100 | mg |
| Phospholipid | 3.0 | g | / | | / | | 2.0 | g |
| EPCS | | | | | | | | |
| Phospholipid | / | | 3.0 | | / | | / | |
| PC-98T | | | | | | | | |
| Phospholipid | / | | / | | 3.0 | g | / | |
| E80 | | | | | | | | |
| Phospholipid | / | | / | | / | | 1.0 | g |
| HSPC | | | | | | | | |
| Cholesterol | 200 | mg | 200 | mg | 200 | mg | 200 | mg |
| Anhydrous | 4 | mL | 4 | mL | 4 | mL | 4 | mL |
| ethanol | | | | | | | | |
| Water for | to 100 | mL | to 100 | mL | to 100 | mL | to 100 | mL |
| injection | | | | | | | | |

### 2.2 Preparation Method

Formula amounts of temsirolimus, phospholipid and cholesterol were weighed, formula amount of anhydrous ethanol was added thereto, and them were dissolved by heating at 60° C. to give an organic phase; formula amount of water for injection was weighed and heated to 60° C. to give an aqueous phase; the organic phase was poured into the aqueous phase with stirring, mixed uniformly to give a crude liposome; the crude liposome was placed in an extruder, extruded to successively through extrusion membranes with pore sizes of 0.2 μm, 0.1 μm, 0.05 μm, and thus the formulation was obtained.

### 1.2.3 Results Evaluation

| | | Encapsulation |
|---|---|---|
| Group | Appearance | efficiency |
| Formulation 1 | opaque, suspension, having obviously | 85.18% |
| | visible microparticles, precipitates | |
| | appeared at the bottom after placement | |
| | or centrifugation | |
| Formulation 2 | opaque, suspension, having obviously | 82.44% |
| | visible microparticles, precipitates | |
| | appeared at the bottom after placement | |
| | or centrifugation | |
| Formulation 3 | opaque, suspension, having obviously | 75.51% |
| | visible microparticles, obvious | |
| | precipitates appeared faster than | |
| | Formulations 1 and 2 | |
| Formulation 4 | opaque, suspension, having obviously | 83.89%. |
| | visible microparticles, precipitates | |
| | appeared at the bottom after placement | |
| | or centrifugation | |

### 3. Fat Emulsion

Investigation of the solubility of temsirolimus in common oils for injection (medium chain oil, soybean oil)

| | Concentration | | | | |
|---|---|---|---|---|---|
| | Medium chain oil | | | | Soybean oil |
| Dissolution time | 40 mg/g | 30 mg/g | 20 mg/g | 10 mg/g | 10 mg/g |
| 60° C., 30 min | Severe | Severe | Severe | Severe | Severe |
| | turbid | turbid | turbid | turbid | turbid |
| 70° C., 30 min | Severe | Severe | Severe | Severe | Severe |
| | turbid | turbid | turbid | turbid | turbid |
| 80° C., 30 min | Severe | Severe | Severe | Severe | Severe |
| | turbid | turbid | turbid | turbid | turbid |

If 10% oil was used, in order to achieve a drug loading of 1 mg/ml, the solubility of the drug in oil also needed to reach 10 mg/g. However, according to the above investigation results on the solubility, the solubilities of temsirolimus in the medium chain oil and soybean oil were both poor, hardly to achieve the preparation of the fat emulsion.

In summary, (1) 1 mg/mL of temsirolimus still cannot be included, even if the application amount of sulfobutyl ether-β-cyclodextrin has reached 50%; (2) the quality of the temsirolimus liposome was poor, such as the deficiencies of having visible microparticles, instability, low encapsulation efficiencies and the like; (3) temsirolimus was added into the medium chain oil or soybean oil at a concentration of 10 mg/g, exhibited a state of severe turbidity and cannot be dissolved after being heated under high temperature. It can be seen that the preparation of temsirolimus into sulfobutyl ether-β-cyclodextrin inclusion complex and fat emulsion was almost impossible. Regarding the temsirolimus liposome, although the preparation quality of the current formulation process was still not good, relatively speaking, the optimization of the formulation process was more feasible.

### Example 2 Criticality of PEGylated Phospholipid on the Development of Temsirolimus Liposome

Liposome was generally consisted of lecithin, or lecithin and cholesterol. However, in the case of temsirolimus, quantum satis PEGylated phospholipid must be added into the formulation. Otherwise, the problems of turbidity and precipitation occur in a very short time and stable liposomes cannot be prepared, no matter how the formulation and the process were adjusted. The typical verification protocol was as follows.

Taking DSPE-PEG2000 as an example:

### 1. Formulation:

| Component | Formulation 1 | | Formulation 2 | | Formulation 3 | | Formulation 4 | | Formulation 5 | | Formulation 6 | |
|---|---|---|---|---|---|---|---|---|---|---|---|---|
| Temsirolimus | 125 | mg | 125 | mg | 125 | mg | 125 | mg | 125 | mg | 125 | mg |
| EPCS | 3.5 | g | 4.5 | g | 5.5 | g | 3.5 | g | 3.5 | g | 3.5 | g |
| Cholesterol | 0.2 | g | 0.2 | g | 0.2 | g | 0.2 | g | 0.2 | g | 0.2 | g |
| DSPE-PEG2000 | / | | / | | / | | 10 | mg | 50 | mg | 100 | mg |
| Ethanol | 4 | mL | 4 | mL | 4 | mL | 4 | mL | 4 | mL | 4 | mL |
| Water for | to 100 | mL | to 100 | mL | to 100 | mL | to 100 | mL | to 100 | mL | to 100 | mL |
| Injection | | | | | | | | | | | | |

### 2. Preparation Process

Formula amounts of temsirolimus, high-purity egg yolk lecithin (EPCS), cholesterol or DSPE-PEG2000 were weighed, formula amount of ethanol was added thereto, and them were dissolved by heating at 50° C. to give an organic phase; formula amount of water for injection was weighed and heated to 50° C. to give an aqueous phase; the organic phase was poured into the aqueous phase with stirring, mixed uniformly to give a crude liposome; the crude liposome was extruded successively through extrusion membranes with pore sizes of 0.2 μm, 0.1 μm, 0.05 μm, and thus the temsirolimus liposome was obtained.

### 3. Test Results

The effect of DSPE-PEG2000 on druggability

| | | Insoluble microparticle | | Encapsulation |
|---|---|---|---|---|
| Group | Appearance | ≥10 μm | ≥25 μm | on efficiency % |
| Formulation 1 | opaque, suspension, having | >10000/mL | >500/mL | 85.18% |
| | obviously visible microparticles, | | | |
| | precipitates appeared at the | | | |
| | bottom after short placement or | | | |
| | centrifugation | | | |
| Formulation 2 | opaque, suspension, having | >10000/mL | >500/mL | 89.44% |
| | obviously visible microparticles, | | | |
| | precipitates appeared at the | | | |
| | bottom after short placement or | | | |
| | centrifugation | | | |
| Formulation 3 | opaque, suspension, having | >9000/mL | >300/mL | 92.01% |
| | obviously visible microparticles, | | | |
| | precipitates appeared at the | | | |
| | bottom after short placement or | | | |
| | centrifugation | | | |
| Formulation 4 | slightly transparent, no | 58/mL | 3/mL | 92.37% |
| | obviously visible microparticles | | | |
| | or precipitates, precipitates | | | |
| | remained invisible after | | | |
| | centrifugation, a small amount | | | |
| | of microparticles being visible | | | |
| | after 24 h of placement | | | |
| Formulation 5 | translucent and homogenous | 17/mL | 0/mL | 96.22% |
| | liquid, microparticles and | | | |
| | precipitates remained invisible | | | |
| | after 24 h of placement or | | | |
| | centrifugation | | | |
| Formulation 6 | translucent and homogenous | 11/mL | 0/mL | 98.92% |
| | liquid, microparticles and | | | |
| | precipitates remained invisible | | | |
| | after 24 h of placement or | | | |
| | centrifugation | | | |

### Results Analysis:

When DSPE-PEG2000 was not included in the formulation, the prepared liposome solution was in a state of suspension, and precipitates were obviously observed at the bottom in a very short time. Even if the application amount of phospholipid was increased to 5.5%, this state still cannot be improved, and a large number of microparticles were shown to be contained therein after the detection of insoluble microparticles; in addition, the results were similar by investigating other various phospholipids, and stable temsirolimus liposome cannot be obtained.

When a small amount of DSPE-PEG2000 was added to the formulation, the appearance of the solution was significantly improved, showing a translucent and homogenous liquid with no precipitates and visible microparticles. A few insoluble microparticles were detected, and microparticles and precipitates remained invisible after 24 h of placement. Accordingly, the stability of the liposome was improved significantly. At the same time, the encapsulation efficiency was significantly higher than that of the phospholipid in the same application amount.

Similarly, DSPE-PEG5000, DPPE-PEG2000 and DPPE-PEG5000 were investigated, and similar results to that of DSPE-PEG2000 were obtained.

It can be seen that the addition of PEGylated phospholipid is of great importance to the temsirolimus liposome, which is the core technical feature of the present invention, and directly affects the druggability of the liposome.

### Example 3 Preparation of Temsirolimus Liposome

0.15 g of temsirolimus, 3.5 g of high-purity egg yolk lecithin (EPCS), 0.125 g of DSPE-PEG2000, 0.03 g of α-tocopherol were weighed, 8.0 g of tert-butanol was added thereto, and them were dissolved by heating at 45° C., placed in a sample plate, and lyophilized to remove the organic vehicle to give a lipid phase; 0.01 g of EDTA-2Na, 75 g of water for injection were weighed, heated to 45° C. and dissolved to give an aqueous phase; the aqueous phase was added to the lipid phase, fully dissolved and dispersed with stirring to give a crude liposome; the crude liposome was placed in an extruder and extruded successively through extrusion membranes with pore sizes of 0.2 μm, 0.1 μm, 0.05 μm, so as to give a liposome solution; 20 g of maltose was weighed, placed in the above liposome solution, and dissolved with stirring, and setting the volume thereof to 100 mL with water for injection; the pH value was adjusted with citric acid, sodium citrate to 5.5; it was filtered and sterilized through a 0.22 μm filter membrane, sub-packaged, lyophilized and sealed, and thus the temsirolimus liposome lyophilized powder injection was obtained.

The average particle size was determined to be 98.8 nm.

### Example 4 Preparation of Temsirolimus Liposome

0.125 g of temsirolimus, 3.5 g of high-purity egg yolk lecithin (EPCS), 0.1 g of cholesterol, 0.125 g of DSPE-PEG2000, 0.03 g of α-tocopherol were weighed, 3.5 g of anhydrous ethanol was added thereto, and them were dissolved by heating at 50° C. to give an organic phase; 75 g of water for injection was weighed and heated to 50° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.2 μm, 0.1 μm, 0.05 μm, so as to give a liposome solution; the liposome solution was subjected to ultrafiltration to remove anhydrous ethanol; 25 g of sucrose was weighed, placed into the above liposome solution, and dissolved with stirring, and setting the volume thereof to 100 mL with water for injection; the pH value was adjusted with citric acid, sodium citrate to 5.5; it was filtered and sterilized through a 0.22 μm filter membrane, sub-packaged, lyophilized and sealed, and thus the temsirolimus liposome lyophilized powder injection was obtained.

The average particle size was determined to be 96.8 nm.

### Example 5 Preparation of Temsirolimus Liposome

0.05 g of temsirolimus, 1.5 g of high-purity egg yolk lecithin (EPCS), 50 mg of DSPE-PEG2000, 0.005 g of α-tocopherol were weighed, quantum satis anhydrous ethanol was added thereto, and them were dissolved by heating at 45° C. to give an organic phase; the organic phase was subjected to rotary evaporation to remove the organic solvent at 45° C., so as to give a lipid phase; 90 g of water for injection was weighed and heated to 45° C., giving an aqueous phase; the aqueous phase was added to the lipid phase to hydrate the lipid components, so as to give a crude liposome; the crude liposome was placed in an extruder and extruded successively through extrusion membranes with pore sizes of 0.2 μm, 0.1 μm, 0.05 μm, so as to give a liposome solution; 5 g of trehalose was weighed, placed into the above liposome solution, and dissolved with stirring, and setting the volume thereof to 100 mL with water for injection; the pH value was adjusted with citric acid, sodium citrate to 5.5; it was filtered and sterilized through a 0.22 μm filter membrane, sub-packaged, lyophilized and sealed, and thus the temsirolimus liposome lyophilized powder injection was obtained.

The average particle size was determined to be 70.6 nm.

### Example 6 Preparation of Temsirolimus Liposome

0.1 g of temsirolimus, 2.0 g of high-purity egg yolk lecithin (EPCS), 0.1 g of cholesterol, 0.1 g of DSPE-PEG5000, 0.01 g of α-tocopherol were weighed, 1.0 g of anhydrous ethanol was added thereto, and them were dissolved by heating at 30° C. to give an organic phase; 15 g of trehalose and 80 g of water for injection were weighed, dissolved by heating to 30° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.2 μm, 0.1 μm, 0.05 μm, so as to give a liposome solution; the volume thereof was set to 100 mL with water for injection; the pH value was adjusted with citric acid to 5.0; it was filtered and sterilized through a 0.22 μm filter membrane, sub-packaged, lyophilized and sealed, and thus the temsirolimus liposome lyophilized powder injection was obtained.

The average particle size was determined to be 78.2 nm.

### Example 7 Preparation of Temsirolimus Liposome

0.1 g of temsirolimus, 2.0 g of hydrogenated soybean phospholipid (HSPC), 0.1 g of cholesterol, 20 mg of DSPE-PEG5000 were weighed, quantum satis dichloromethane was added thereto, and them were dissolved by heating at 60° C. to give an organic phase; the organic phase was subjected to rotary evaporation to remove the organic solvent at 40° C., so as to give a lipid phase; 0.02 g of EDTA-2Na, 20 g of sucrose and 75 g of water for injection were weighed, dissolved by heating to 60° C., so as to give an aqueous phase; the aqueous phase was added to the lipid phase to hydrate the lipid components, giving a crude liposome; the crude liposome was placed in an extruder and extruded successively through extrusion membranes with pore sizes of 0.2 μm, 0.1 μm, 0.05 μm, so as to give a liposome solution; the volume thereof was set to 100 mL with water for injection; the pH value was adjusted with citric acid, sodium citrate to 6.0; it was filtered and sterilized through a 0.22 μm filter membrane, sub-packaged, lyophilized and sealed, and thus the temsirolimus liposome lyophilized powder injection was obtained.

The average particle size was determined to be 90.8 nm.

### Example 8 Preparation of Temsirolimus Liposome

0.075 g of temsirolimus, 1.5 g of high-purity egg yolk lecithin (EPCS), 0.1 g of DPPE-PEG2000 were weighed, 3.0 g of tert-butanol was added thereto, and them were dissolved by heating at 55° C. to give an organic phase; the organic phase was placed in a sample plate, and lyophilized to remove the organic vehicle, so as to give a lipid phase; 0.2 g of thioglycerol, 0.02 g of EDTA-2Na, 7 g of glucose, 15 g of sorbitol, 70 g of water for injection were weighed, and dissolved by heating to 55° C., so as to give an aqueous phase; the aqueous phase was added to the lipid phase, fully dissolved and dispersed with stirring, giving a crude liposome; the crude liposome was homogenized and emulsified by placing it in a high-pressure homogenizer, so as to give a liposome solution; the volume thereof was set to 100 m L with water for injection; the pH value was adjusted with citric acid, sodium citrate to 6.0; it was filtered and sterilized through a 0.22 μm filter membrane, sub-packaged, lyophilized and sealed, and thus the temsirolimus liposome lyophilized powder injection was obtained.

The average particle size was determined to be 87.7 nm.

### Example 9 Preparation of Temsirolimus Liposome

0.125 g of temsirolimus, 3.0 g of high-purity egg yolk lecithin (EPCS), 0.1 g of cholesterol, 0.15 g of DSPE-PEG2000, 0.03 g of α-tocopheryl succinate were weighed, 2.0 g of anhydrous ethanol was added thereto, and them were dissolved by heating at 35° C. to give an organic phase; 0.01 g of EDTA-2Na, 5 g of glucose, and 70 g of water for injection were weighed, and dissolved by heating to 35° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.2 μm, 0.1 μm, 0.05 μm, so as to give a liposome solution; 8 g of glucose and 10 g of mannitol were weighed, placed into the above liposome solution, dissolved with stirring, and the volume thereof was set to 100 mL with water for injection; the pH value was adjusted with hydrochloric acid to 3.0; it was filtered and sterilized through a 0.22 μm filter membrane, sub-packaged, lyophilized and sealed, and thus the temsirolimus liposome lyophilized powder injection was obtained.

The average particle size was determined to be 89.8 nm.

### Example 10 Preparation of Temsirolimus Liposome

0.125 mg of temsirolimus, 3.5 g of high-purity egg yolk lecithin (PC-98T), 0.1 g of cholesterol, 0.3 g of DSPE-PEG2000, 0.02 g of α-tocopheryl acetate were weighed, 4.0 g of propanediol was added thereto, and them were dissolved by heating at 60° C. to give an organic phase; 80 g of water for injection was weighed, and dissolved by heating to 60° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.2 μm, 0.1 μm, 0.05 μm, so as to give a liposome solution; the liposome solution was subjected to ultrafiltration to remove propanediol; 10 g of sucrose was weighed, placed into the above liposome solution, dissolved with stirring, and the volume thereof was set to 100 mL with water for injection; the pH value was adjusted with citric acid to 4.5; it was filtered and sterilized through a 0.22 μm filter membrane, sub-packaged, lyophilized and sealed, and thus the temsirolimus liposome lyophilized powder injection was obtained.

The average particle size was determined to be 94.8 nm.

### Example 11 Preparation of Temsirolimus Liposome

0.15 g of temsirolimus, 3.5 g of high-purity egg yolk lecithin (EPCS), 0.3 g of cholesterol, 0.125 g of DPPE-PEG2000, 0.02 g of α-tocopheryl acetate were weighed, 3.0 g of tert-butanol and 2.0 g of methanol were added thereto, and them were dissolved by heating at 55° C. to give an organic phase; the organic phase was placed in a sample plate, and lyophilized to remove the organic vehicle, so as to give a lipid phase; 0.02 g of EDTA-2Na, 70 g of water for injection were weighed, and dissolved by heating at 55° C. to give an aqueous phase; the aqueous phase was added to the lipid phase, fully dissolved and dispersed with stirring to give a crude liposome; the crude liposome was homogenized and emulsified by placing it in a high-pressure homogenizer, so as to give a liposome solution; 7 g of glucose and 15 g of sorbitol were weighed, placed into the above liposome solution, dissolved with stirring, and the volume thereof was set to 100 mL with water for injection; the pH value was adjusted with acetic acid, sodium acetate to 6.0; it was filtered and sterilized through a 0.22 μm filter membrane, sub-packaged, lyophilized and sealed, and thus the temsirolimus liposome lyophilized powder injection was obtained.

The average particle size was determined to be 97.7 nm.

### Example 12 Preparation of Temsirolimus Liposome

0.15 g of temsirolimus, 2.5 g of high-purity egg yolk lecithin (PC-98T), 0.5 g of 1-palmitoyl-2-oleoyl-sn-glycero-β-phosphocholine (POPC), 0.25 g of cholesterol, 0.2 g of DSPE-PEG2000 and 0.1 g of adipic acid were weighed, 4.5 g of anhydrous ethanol was added thereto, and them were dissolved by heating at 60° C. to give an organic phase; 0.1 g of glutathione, 75 g of water for injection were weighed, and dissolved by heating to 60° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.4 μm, 0.2 μm, 0.1 μm and 0.05 μm, so as to give a liposome solution; the liposome solution was subjected to a scraper film evaporator to remove anhydrous ethanol; 20 g of xylitol was weighed, placed into the above liposome solution, dissolved with stirring, and the volume thereof was set to 100 mL with water for injection; the pH value was adjusted with disodium hydrogen phosphate, sodium dihydrogen phosphate to 6.5; it was filtered and sterilized through a 0.22 μm filter membrane, sub-packaged, lyophilized and sealed, and thus the temsirolimus liposome lyophilized powder injection was obtained.

The average particle size was determined to be 102.0 nm.

### Example 13 Preparation of Temsirolimus Liposome

0.15 g of temsirolimus, 2.0 g of high-purity egg yolk lecithin (EPCS), 1.0 g of dipalmitoyl phosphatidylcholine (DPPC), 0.2 g of DPPE-PEG2000 and 0.4 g of di-tert-butyl p-cresol were weighed, 3.0 g of anhydrous ethanol and 2.0 g of tert-butanol were added thereto, and them were dissolved by heating at 65° C. to give an organic phase; 0.05 g of ethylenediamine tetraacetic acid, 75 g of water for injection were weighed and heated to 65° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.4 μm, 0.2 μm, 0.1 μm and 0.05 μm, so as to give a liposome solution; 10 g of trehalose and 10 g of maltose were weighed, placed into the above liposome solution, and dissolved with stirring, and setting the volume thereof to 100 mL with water for injection; the pH value was adjusted with phosphoric acid to 4.0; it was filtered and sterilized through a 0.22 μm filter membrane, sub-packaged, lyophilized and sealed, and thus the temsirolimus liposome lyophilized powder injection was obtained.

The average particle size was determined to be 105.1 nm.

### Example 14 Preparation of Temsirolimus Liposome

0.125 g of temsirolimus, 2.0 g of high-purity egg yolk lecithin (EPCS), 1.0 g of distearoyl phosphatidylcholine (DSPC), 0.1 g of DPPE-PEG5000 were weighed, and dissolved by adding quantum satis anhydrous ethanol thereto, so as to give an organic phase; the organic phase was subjected to rotary evaporation to remove the organic solvent at 45° C., giving a lipid phase; 0.5 g of cysteine and 85 g of water for injection were weighed, heated to 45° C., dissolved to give an aqueous phase; the aqueous phase was added to the lipid phase to hydrate the lipid components, so as to give a crude liposome; the crude liposome was placed in an extruder and extruded successively through extrusion membranes with pore sizes of 0.2 μm, 0.1 μm, 0.05 μm, so as to give a liposome solution; 10 g of maltose was weighed, placed into the above liposome solution, and dissolved with stirring, and setting the volume thereof to 100 mL with water for injection; the pH value was adjusted with dipotassium hydrogen phosphate, potassium dihydrogen phosphate to 7.0; it was filtered and sterilized through a 0.22 μm filter membrane, sub-packaged, lyophilized and sealed, and thus the temsirolimus liposome lyophilized powder injection was obtained.

The average particle size was determined to be 94.6 nm.

### Example 15 Preparation of Temsirolimus Liposome

0.175 g of temsirolimus, 2.5 g of high-purity egg yolk lecithin (PC-98T), 1.0 g of dierucoyl phosphatidylcholine (DEPC), 0.15 g of cholesterol, 0.2 g of DSPE-PEG1000, 0.2 g of propyl gallate were weighed, 4.0 g of anhydrous ethanol was added thereto, and them were dissolved by heating at 55° C. to give an organic phase; 0.3 g of maleic acid, 75 g of water for injection were weighed, and dissolved by heating to 55° C., so as to give an aqueous phase; the organic phase was poured into the aqueous phase with stirring, and mixed uniformly to give a crude liposome; the crude liposome was homogenized and emulsified by placing it in a high-pressure homogenizer, and then placed in an extruder and extruded successively through extrusion membranes with pore sizes of 0.1 μm and 0.05 μm, so as to give a liposome solution; 15 g of maltose, 5 g of sucrose and 5 g of threonine were weighed, placed into the above liposome solution, and dissolved with stirring, and setting the volume thereof to 100 mL with water for injection; the pH value was adjusted with triethylamine to 8.0; it was filtered and sterilized through a 0.22 μm filter membrane, sub-packaged, lyophilized and sealed, and thus the temsirolimus liposome lyophilized powder injection was obtained.

The average particle size was determined to be 106.8 nm.

### Example 16 Preparation of Temsirolimus Liposome

0.2 g of temsirolimus, 3.0 g of soybean phospholipid, 0.5 g of dioleoyl phosphatidylcholine (DOPC), 0.2 g of DPPE-PEG5000 were weighed, 4.0 g of propanediol was added thereto, and them were dissolved by heating at 70° C. to give an organic phase; 0.5 g of malic acid, 70 g of water for injection were weighed, and dissolved by heating to 70° C., so as to give an aqueous phase; the organic phase was poured into the aqueous phase with stirring, and mixed uniformly to give a crude liposome; the crude liposome was placed in an extruder and extruded successively through extrusion membranes with pore sizes of 0.4 μm, 0.2 μm, 0.1 μm and 0.05 μm, so as to give a liposome solution; 14 g of glucose and 11 g of mannitol were weighed, placed into the above liposome solution, and dissolved with stirring, and setting the volume thereof to 100 mL with water for injection; the pH value was adjusted with citric acid, sodium citrate to 6.0; it was filtered and sterilized through a 0.22 μm filter membrane, sub-packaged, lyophilized and sealed, and thus the temsirolimus liposome lyophilized powder injection was obtained.

The average particle size was determined to be 108.9 nm.

### Example 17 Preparation of Temsirolimus Liposome

0.25 g of temsirolimus, 4.0 g of high-purity soybean lecithin (S100), 0.1 g of cholesterol, 0.25 g of DSPE-PEG8000, 0.04 g of α-tocopherol were weighed, 8.0 g of tert-butanol and 2.0 g of methanol were added thereto, and them were dissolved by heating at 50° C. to give an organic phase; the organic phase was placed in a sample plate, and lyophilized to remove the organic vehicle, giving a lipid phase; 0.5 g of glutamic acid, 10 g of sucrose, 70 g of water for injection were weighed, and dissolved by heating to 50° C., so as to give an aqueous phase; the aqueous phase was added to the lipid phase, fully dissolved and dispersed with stirring to give a crude liposome; the crude liposome was placed in an extruder and extruded successively through extrusion membranes with pore sizes of 0.4 μm, 0.2 μm, 0.1 μm and 0.05 μm, so as to give a liposome solution; 15 g of sucrose was weighed, placed into the above liposome solution, and dissolved with stirring, and setting the volume thereof to 100 mL with water for injection; the pH value was adjusted with citric acid to 5.0; it was filtered and sterilized through a 0.22 μm filter membrane, sub-packaged, lyophilized and sealed, and thus the temsirolimus liposome lyophilized powder injection was obtained.

The average particle size was determined to be 112.4 nm.

### Example 18 Preparation of Temsirolimus Liposome

0.5 g of temsirolimus, 6.0 g of high-purity egg yolk lecithin (PC-98T), 0.4 g of cholesterol and 0.5 g of DSPE-PEG6000 were weighed, 5.0 g of anhydrous ethanol was added thereto, and them were dissolved by heating at 50° C. to give an organic phase; 0.03 g of EDTA-2Na, 0.6 g of glutathione, 55 g of water for injection were weighed, and dissolved by heating to 50° C., giving an aqueous phase; the organic phase was poured into the aqueous phase with stirring, and mixed uniformly to give a crude liposome; the crude liposome was placed in an extruder and extruded successively through extrusion membranes with pore sizes of 0.8 μm, 0.4 μm, 0.2 μm, 0.1 μm and 0.05 μm, so as to give a liposome solution; the liposome solution was subjected to a scraper film evaporator to remove anhydrous ethanol; 20 g of maltose, 15 g of sucrose were weighed, placed into the above liposome solution, and dissolved with stirring, and setting the volume thereof to 100 mL with water for injection; the pH value was adjusted with sodium hydroxide to 8.0; it was filtered and sterilized through a 0.22 μm filter membrane, sub-packaged, lyophilized and sealed, and thus the temsirolimus liposome lyophilized powder injection was obtained.

The average particle size was determined to be 125.5 nm.

### Example 19 Preparation of Temsirolimus Liposome

0.7 g of temsirolimus, 8.0 g of high-purity egg yolk lecithin (PC-98T), 0.5 g of cholesterol, 0.7 g of DSPE-PEG4000, 0.08 g of α-tocopherol were weighed, 10.0 g of tert-butanol and 5.0 g of acetonitrile were added thereto, and them were dissolved by heating at 60° C. to give an organic phase; the organic phase was placed in a sample plate, and lyophilized to remove the organic vehicle, giving a lipid phase; 0.1 g of ascorbic acid, 50 g of water for injection were weighed, and dissolved by heating to 60° C., so as to give an aqueous phase; the aqueous phase was added to the lipid phase, fully dissolved and dispersed with stirring, so as to give a crude liposome; the crude liposome was homogenized and emulsified by placing it in a high-pressure homogenizer, and then placed in an extruder and extruded successively through extrusion membranes with pore sizes of 0.2 μm, 0.1 μm and 0.05 μm, so as to give a liposome solution; 20 g of maltose and 20 g of trehalose were weighed, placed into the above liposome solution, and dissolved with stirring, and setting the volume thereof to 100 mL with water for injection; the pH value was adjusted with acetic acid, sodium acetate to 5.5; it was filtered and sterilized through a 0.22 μm filter membrane, sub-packaged, lyophilized and sealed, and thus the temsirolimus liposome lyophilized powder injection was obtained.

The average particle size was determined to be 130.2 nm.

### Example 20 Preparation of Temsirolimus Liposome

0.3 g of temsirolimus, 2.5 g of high-purity egg yolk lecithin (PC-98T), 1.0 g of dimyristoyl phosphatidylcholine (DMPC), 0.2 g of cholesterol, 0.2 g of DPPE-PEG1000, 0.1 g of tert-butyl hydroxyanisole were weighed, quantum satis dichloromethane was added thereto, and them were dissolved by heating at 40° C. to give an organic phase; the organic phase was subjected to rotary evaporation to remove the organic solvent at 40° C., giving a lipid phase; 0.1 g of succinic acid, 10 g of maltose, 13 g of trehalose and 70 g of water for injection were weighed, and dissolved by heating to 40° C., so as to give an aqueous phase; the aqueous phase was added to the lipid phase to hydrate the lipid components, so as to give a crude liposome; the crude liposome was homogenized and emulsified by placing it in a high-pressure homogenizer to give a liposome solution; setting the volume thereof to 100 mL with water for injection; the pH value was adjusted with citric acid to 5.0; it was filtered and sterilized through a 0.22 μm filter membrane, sub-packaged, lyophilized and sealed, and thus the temsirolimus liposome lyophilized powder injection was obtained.

The average particle size was determined to be 115.7 nm.

### Example 21 Preparation of Temsirolimus Liposome

0.6 g of temsirolimus, 8.0 g of high-purity egg yolk lecithin (PC-98T), 0.4 g of cholesterol, 0.5 g of DSPE-PEG2000, 0.15 g of α-tocopherol were weighed, 5.0 g of anhydrous ethanol was added thereto, and them were dissolved by heating at 55° C. to give an organic phase; 70 g of water for injection was weighed, and dissolved by heating to 55° C., so as to give an aqueous phase; the organic phase was poured into the aqueous phase with stirring, and mixed uniformly to give a crude liposome; the crude liposome was placed in an extruder and extruded successively through extrusion membranes with pore sizes of 1.0 μm, 0.6 μm, 0.4 μm, 0.2 μm, 0.1 μm and 0.05 μm, so as to give a liposome solution; the liposome solution was subjected to ultrafiltration to remove anhydrous ethanol; 10 g of maltose and 10 g of erythritol were weighed, placed into the above liposome solution, and dissolved with stirring, and setting the volume thereof to 100 mL with water for injection; the pH value was adjusted with citric acid, sodium citrate to 6.0; it was filtered and sterilized through a 0.22 μm filter membrane, sub-packaged, lyophilized and sealed, and thus the temsirolimus liposome lyophilized powder injection was obtained.

The average particle size was determined to be 125.6 nm.

### Example 22 Preparation of Temsirolimus Liposome

0.8 g of temsirolimus, 10.0 g of high-purity egg yolk lecithin (PC-98T), 1.0 g of cholesterol, 0.9 g of DPPE-PEG8000 were weighed, quantum satis dichloromethane was added thereto, and them were dissolved by heating at 40° C. to give an organic phase; the organic phase was subjected to rotary evaporation to remove the organic solvent at 40° C., so as to give a lipid phase; 0.6 g of thioglycerol, 0.2 g of sodium metabisulfite and 55 g of water for injection were weighed, and dissolved by heating to 50° C., giving an aqueous phase; the aqueous phase was added to the lipid phase to hydrate the lipid components, so as to give a crude liposome; the crude liposome was placed in an extruder and extruded successively through extrusion membranes with pore sizes of 2.0 μm, 1.0 μm, 0.6 μm, 0.4 μm, 0.2 μm, 0.1 μm and 0.05 μm, so as to give a liposome solution; 20 g of sucrose, 10 g of maltose were weighed, placed into the above liposome solution, and dissolved with stirring, and setting the volume thereof to 100 mL with water for injection; the pH value was adjusted with citric acid to 5.0; it was filtered and sterilized through a 0.22 μm filter membrane, sub-packaged, lyophilized and sealed, and thus the temsirolimus liposome lyophilized powder injection was obtained.

The average particle size was determined to be 133.6 nm.

### Example 23 Preparation of Temsirolimus Liposome

0.4 g of temsirolimus, 3.0 g of high-purity egg yolk lecithin (EPCS), 3.0 g of high-purity egg yolk lecithin (98T), 0.2 g of cholesterol, 0.3 g of DSPE-PEG2000, 0.05 g of α-tocopheryl acetate were weighed, 5.0 g of tert-butanol and 2.0 g of propanediol were added thereto, and them were dissolved by heating at 60° C. to give an organic phase; the organic phase was placed in a sample plate, and lyophilized to remove the organic vehicle, so as to give a lipid phase; 0.1 g of sodium bisulfite, 15 g of sucrose, 5 g of xylitol, 5 g of threonine, 70 g of water for injection were weighed, and dissolved by heating to 60° C., giving an aqueous phase; the aqueous phase was added to the lipid phase, fully dissolved and dispersed with stirring, so as to give a crude liposome; the crude liposome was placed in an extruder and extruded successively through extrusion membranes with pore sizes of 0.6 μm, 0.4 μm, 0.2 μm, 0.1 μm and 0.05 μm, so as to give a liposome solution; the volume thereof was set to 100 mL with water for injection; the pH value was adjusted with citric acid to 4.0; it was filtered and sterilized through a 0.22 μm filter membrane, sub-packaged, lyophilized and sealed, and thus the temsirolimus liposome lyophilized powder injection was obtained.

The average particle size was determined to be 124.0 nm.

### Example 24 Preparation of Temsirolimus Liposome

1.0 g of temsirolimus, 10.0 g of high-purity egg yolk lecithin (PC-98T), 0.4 g of cholesterol and 1.0 g of DSPE-PEG2000 were weighed, 15.0 g of tert-butanol and 5.0 g of acetonitrile were added thereto, and them were dissolved by heating at 60° C. to give an organic phase; the organic phase was placed in a sample plate, and lyophilized to remove the organic vehicle, so as to give a lipid phase; 0.8 g of thioglycerol, 0.2 g of glutathione, 10 g of maltose and 65 g of water for injection were weighed, and dissolved by heating to 60° C., giving an aqueous phase; the aqueous phase was added to the lipid phase, fully dissolved and dispersed with stirring, so as to give a crude liposome; the crude liposome was placed in an extruder and extruded successively through extrusion membranes with pore sizes of 2.0 μm, 1.0 μm, 0.8 μm, 0.4 μm, 0.2 μm, 0.1 μm and 0.05 μm, so as to give a liposome solution; 10 g of maltose was weighed, placed into the above liposome solution, and dissolved by stirring, and setting the volume thereof to 100 mL with water for injection; the pH value was adjusted with acetic acid, sodium acetate to 5.5; it was filtered and sterilized through a 0.22 μm filter membrane, sub-packaged, lyophilized and sealed, and thus the temsirolimus liposome lyophilized powder injection was obtained.

The average particle size was determined to be 138.9 nm.

### Example 25 Preparation of Temsirolimus Liposome

0.2 g of temsirolimus, 3.5 g of high-purity egg yolk lecithin (EPCS), 0.2 g of cholesterol, 0.15 g of DSPE-PEG2000 and 0.02 g of α-tocopherol were weighed, 8.0 g of tert-butanol and 2.0 g of ethanol were added thereto, and them were dissolved by heating at 40° C. to give an organic phase; the organic phase was placed in a sample plate, and lyophilized to remove the organic vehicle, so as to give a lipid phase; 0.2 g of cysteine and 75 g of water for injection were weighed, and dissolved by heating to 40° C., giving an aqueous phase; the aqueous phase was added to the lipid phase, fully dissolved and dispersed with stirring, so as to give a crude liposome; the crude liposome was placed in an extruder and extruded successively through extrusion membranes with pore sizes of 0.4 μm, 0.2 μm, 0.1 μm and 0.05 μm, so as to give a liposome solution; 20 g of trehalose was weighed, placed into the above liposome solution, and dissolved by stirring, and setting the volume thereof to 100 mL with water for injection; the pH value was adjusted with disodium hydrogen phosphate, sodium dihydrogen phosphate to 7.0; it was filtered and sterilized through a 0.22 μm filter membrane, sub-packaged, lyophilized and sealed, and thus the temsirolimus liposome lyophilized powder injection was obtained.

The average particle size was determined to be 105.6 nm.

### Example 26 Preparation of Temsirolimus Liposome

0.25 g of temsirolimus, 4.0 g of high-purity egg yolk lecithin (PC-98T), 0.2 g of DSPE-PEG2000, 0.03 g of α-tocopherol were weighed, quantum satis chloroform and methanol were added thereto, and them were dissolved by heating at 35° C. to give an organic phase; the organic phase was subjected to rotary evaporation to remove the organic solvents at 35° C., so as to give a lipid phase; 0.01 g of EDTA-2Na and 70 g of water for injection were weighed, and dissolved by heating to 35° C., giving an aqueous phase; the aqueous phase was added to the lipid phase to hydrate the lipid components, so as to give a crude liposome; the crude liposome was homogenized and emulsified by placing it in a high-pressure homogenizer, and then placed in an extruder and extruded through extrusion membrane with pore size of 0.05 μm, so as to give a liposome solution; 25 g of maltose was weighed, placed into the above liposome solution, and dissolved with stirring, and setting the volume thereof to 100 mL with water for injection; the pH value was adjusted with citric acid, sodium citrate to 6.5; it was filtered and sterilized through a 0.22 μm filter membrane, sub-packaged, lyophilized and sealed, and thus the temsirolimus liposome lyophilized powder injection was obtained.

The average particle size was determined to be 108.8 nm.

### Example 27 Preparation of Temsirolimus Liposome

0.1 g of temsirolimus, 2.0 g of distearoyl phosphatidylcholine (DSPC), 0.1 g of cholesterol, 0.1 g of DSPE-PEG2000 were weighed, quantum satis chloroform and acetonitrile were added thereto, and them were dissolved by heating at 35° C. to give an organic phase; the organic phase was subjected to rotary evaporation to remove the organic solvents at 35° C., so as to give a lipid phase; 85 g of water for injection was weighed, and heated to 60° C., giving an aqueous phase; the aqueous phase was added to the lipid phase to hydrate the lipid components, so as to give a crude liposome; the crude liposome was placed in an extruder and extruded successively through extrusion membranes with pore sizes of 0.2 μm, 0.1 μm and 0.05 μm, so as to give a liposome solution; 10 g of sucrose was weighed, placed into the above liposome solution, and dissolved with stirring, and setting the volume thereof to 100 mL with water for injection; the pH value was adjusted with hydrochloric acid to 5.5; it was filtered and sterilized through a 0.22 μm filter membrane, sub-packaged, lyophilized and sealed, and thus the temsirolimus liposome lyophilized powder injection was obtained.

The average particle size was determined to be 90.1 nm.

### Example 28 Investigation on the Stability of Temsirolimus Liposome

Samples of Examples 3, 5, 7, 18 and 24 were taken respectively, and placed at 40° C. for 30 days to investigate the stabilities thereof. As indicators, particle size, encapsulation efficiency and drug content were evaluated by sampling on day 0, 10, 20 and 30, respectively. The results were shown in the following Table 1. It can be seen that the temsirolimus liposome of the present invention has excellent stability.

TABLE 1
30-day stability test results for temsirolimus liposome at 40° C.

| | | | | | Example | Example |
|---|---|---|---|---|---|---|
| | | Example 3 | Example 5 | Example 7 | 18 | 24 |
| Particle size | Day 0 | 98.8 nm | 70.6 nm | 90.8 nm | 125.5 nm | 138.9 nm |
| | Day | 97.3 nm | 72.0 nm | 89.2 nm | 128.3 nm | 135.8 nm |
| | 10 | | | | | |
| | Day | 101.8 nm | 72.3 nm | 90.7 nm | 127.0 nm | 134.9 nm |
| | 20 | | | | | |
| | Day | 99.3 nm | 70.9 nm | 92.1 nm | 127.3 nm | 137.2 nm |
| | 30 | | | | | |
| Encapsulation | Day 0 | 98.5% | 99.0% | 97.2% | 96.8% | 96.0% |
| efficiency | Day | 97.2% | 98.2% | 97.8% | 97.6% | 95.8% |
| | 10 | | | | | |
| | Day | 98.1% | 98.0% | 98.5% | 97.9% | 96.9% |
| | 20 | | | | | |
| | Day | 97.9% | 98.7% | 98.0% | 96.3% | 96.0% |
| | 30 | | | | | |
| Drug content | Day 0 | 100% | 100% | 100% | 100% | 100% |
| | Day | 100.22% | 99.80% | 100.72% | 100.02% | 99.37% |
| | 10 | | | | | |
| | Day | 99.69% | 99.49% | 100.09% | 99.34% | 99.80% |
| | 20 | | | | | |
| | Day | 99.92% | 101.10% | 99.31% | 99.36% | 99.27% |
| | 30 | | | | | |

Note:
The drug content of the sample in each Example on day 0 was recorded as 100%, and the drug content thereafter was the percentage relative to that on day 0.

What is claimed is:

1. A temsirolimus liposome composition, characterized in that, the composition is formulated by the following components in weight ratios:

| | | |
|---|---|---|
| Temsirolimus | 0.5-10 | parts |
| Phospholipid | 10-100 | parts |
| PEGylated phospholipid | 0.2-10 | parts |
| Cholesterol | 0-10 | parts |
| Stabilizer | 0-10 | parts |
| Lyoprotectant | 50-400 | parts |
| | |
|---|---|
| Vehicle | q.s. |

2. The temsirolimus liposome composition according to claim 1, characterized in that, the composition is formulated by the following components in weight ratios:

| | | |
|---|---|---|
| Temsirolimus | 0.5-7 | parts |
| Phospholipid | 15-80 | parts |
| PEGylated phospholipid | 0.5-8 | parts |
| Cholesterol | 0-5 | parts |
| Stabilizer | 0.05-8 | parts |
| Lyoprotectant | 100-400 | parts |
| | |
|---|---|
| Vehicle | q.s. |

3. The temsirolimus liposome composition according to claim 1, characterized in that, the composition is formulated by the following components in weight ratios:

| | | |
|---|---|---|
| Temsirolimus | 1-5 | parts |
| Phospholipid | 15-60 | parts |
| PEGylated phospholipid | 1-5 | parts |
| Cholesterol | 0-4 | parts |
| Stabilizer | 0.1-5 | parts |
| Lyoprotectant | 100-350 | parts |
| | |
|---|---|
| Vehicle | q.s. |

4. The temsirolimus liposome composition according to claim 1, characterized in that, the phospholipid is selected from one or two or more of high-purity egg yolk lecithin, high-purity soybean lecithin, hydrogenated soybean lecithin, hydrogenated egg yolk lecithin, dipalmitoyl phosphatidylcholine (DPPC), distearoyl phosphatidylcholine (DSPC), dierucoyl phosphatidylcholine (DEPC), dioleoyl phosphatidylcholine (DOPC), dimyristoyl phosphatidylcholine (DMPC), 1-palmitoyl-2-oleoyl-sn-glycero-β-phosphocholine (POPC), phosphatidylcholine, egg yolk lecithin, soybean lecithin, phosphatidylserine, phosphatidylethanolamine and sphingomyelin, preferably high-purity egg yolk lecithin.

5. The temsirolimus liposome composition according to claim 1, characterized in that, the PEGylated phospholipid is selected from 1,2-distearoyl-sn-glycero-β-phosphoethanolamine-N-[methoxy(polyethylene glycol)] (DSPE-PEG) and/or dipalmitoyl phosphoethanolamine-PEG (DPPE-PEG), preferably 1,2-distearoyl-sn-glycero-β-phosphoethanolamine-N-[methoxy(polyethylene glycol)] (DSPE-PEG); wherein the molecular weight of polyethylene glycol is selected from 1000-8000, preferably 2000-5000.

6. The temsirolimus liposome composition according to claim 1, characterized in that, the stabilizer comprises a chelating agent and/or an antioxidant selected from one or two or more of ethylenediamine tetraacetic acid and salts thereof, α-tocopherol, α-tocopheryl succinate, α-tocopheryl acetate, citric acid, glycine, glutamic acid, succinic acid, adipic acid, malic acid, maleic acid, ascorbic acid, sodium sulfite, sodium bisulfite, sodium metabisulfite, glutathione, cysteine, thioglycerol, tert-butyl hydroxyanisole, di-tert-butyl p-cresol and propyl gallate, preferably α-tocopherol, ethylenediamine tetraacetic acid and salts thereof.

7. The temsirolimus liposome composition according to claim 1, characterized in that, the lyoprotectant is selected from one or two or more of maltose, trehalose, sucrose, mannitol, lactose, glucose, sorbitol, xylitol, erythritol and threonine, preferably sucrose, maltose and trehalose.

8. The temsirolimus liposome composition according to claim 1, characterized in that, 0-40 parts of propanediol, a cosolvent, may further be added as required.

9. A preparation method of the temsirolimus liposome composition according to claim 1, characterized in that, the steps thereof are as follows:
taking temsirolimus, a phospholipid, a PEGylated phospholipid, cholesterol and a stabilizer, adding quantum satis organic vehicle thereto, and dissolving them by heating at 25-75° C. to give an organic phase;
weighing quantum satis water for injection, and heating it to 25-75° C., so as to give an aqueous phase;
pouring the organic phase into the aqueous phase with stirring, and mixing uniformly to give a crude liposome;
emulsifying the crude liposome, which can be homogenized and emulsified by placing it in a high-pressure homogenizer, or extruded successively through extrusion membranes with different pore sizes by placing it in an extruder, or extruded after high-pressure homogenization, so as to give a liposome solution;
weighing a formula amount of a lyoprotectant, placing it in the liposome solution, dissolving it with stirring, and setting the volume thereof to full volume with water for injection;
adjusting pH value with a pH regulator;
filtering and sterilizing through a 0.22 μm filter membrane, sub-packaging, lyophilizing and sealing, and thus obtaining temsirolimus liposome lyophilized powder injection, wherein the organic vehicle is selected from one or two or more of anhydrous ethanol, propanediol and tert-butanol.

10. A preparation method of the temsirolimus liposome composition according to claim 1, characterized in that, the steps thereof are as follows:
taking temsirolimus, a phospholipid, a PEGylated phospholipid, cholesterol and a stabilizer, adding quantum satis organic vehicle thereto, and dissolving them by heating at 25-75° C. to give an organic phase;
subjecting the organic phase to a rotary evaporation to remove the organic vehicle at 25-75° C., or placing the organic phase in a sample plate and lyophilizing it to give a lipid phase;
weighing quantum satis water for injection and heating it to 25-75° C., so as to give an aqueous phase;
adding the aqueous phase to the lipid phase, and stirring them to give a crude liposome;
emulsifying the crude liposome, which can be homogenized and emulsified by placing it in a high-pressure homogenizer, or extruded successively through extrusion membranes with different pore sizes by placing it in an extruder, or extruded after high-pressure homogenization, so as to give a liposome solution;
weighing a formula amount of a lyoprotectant, placing it in the liposome solution, dissolving it with stirring and setting the volume thereof to full volume with water for injection;
adjusting pH value with a pH regulator;
filtering and sterilizing through a 0.22 μm filter membrane, sub-packaging, lyophilizing and sealing, and thus obtaining temsirolimus liposome lyophilized powder injection, wherein the organic vehicle is selected from one or two or more of ethanol, dichloromethane, chloroform, methanol, acetonitrile, tert-butanol, ethylpropanediol and methanol.
